(19) United States
(12) Patent Application Publication
(10) Pub. No.: US 2005/0130163 A1
(43) Pub. Date: Jun. 16, 2005

(54) Serrs reactive particles

(76) Inventors: William Ewen Smith, Glasgow (GB); Duncan Graham, Edinburgh (GB); Peter Cormack, Glasgow (GB); Ailie McCabe, Glasgow (GB)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/507,717

(22) PCT Filed: Jul. 11, 2003
(86) PCT No.: PCT/GB03/03009

(30) Foreign Application Priority Data
Jul. 12, 2002 (GB) 0216197.4

(51) Int. Cl. C12Q 1/68; G01N 33/553

(52) U.S. Cl. 435/6; 436/526

(57) ABSTRACT

The present invention relates to the provision of SERRS active polymer beads and a method of their production for use in detecting targect molecules, as well as methods of detecting target molecules.

The present invention relates to the provision of SERRS active polymer beads and a method of their production for use in detecting target molecules, as well as methods of detecting target molecules.

Recent developments in modern molecular biology have highlighted the need for simultaneous in-situ recognition of a large number of bioanalytes. Among the fields for which there is a requirement is DNA detection, proteomics, cell and antibody recognition, and drug discovery.

Related technologies which involve particles and use detection techniques other than SERRS, include the use of “quantum dots” and the use of the change in surface plasmon resonance which can be achieved by association of gold particles. Quantum dots consist of nanoscale particles of cadmium sulphide/selenide, a well known pigment, which can be modified so that a bioanalyte can be attached. The advantage of this technique is that a single particle gives intense emission compared to a single molecule and the nature of the particles is such that a wide range of emission frequencies can be achieved by altering the ratio of sulphide to selenide.

In the surface plasmon resonance approach, as described in Storhoff, J. J., Elghanian, R., Mucic, R. C., Mirkin, C. A., Letsinger, R. L., J. Am. Chem. Soc 1998, 120, 1959-1964 two gold nanoparticles modified on the surface with specific oligonucleotides are used. Both particles recognise and hybridise a specific strand of DNA which is the analyte. This brings the gold particles into close proximity and the change in frequency of the surface plasmon resonance is detected. However, such techniques do not offer the combined multiplexing, robustness and simplicity which would be desired.

SERRS can uniquely provide the selective labelling chemistry to develop the new types of assays required. It has a sensitivity equivalent to or better than fluorescence but the key advantage is that each SERRS dye used as a label gives a unique fingerprint, which can be recognized in a dye mixture without separation. There are now about 50 specially designed dyes for SERRS, each of which gives a unique spectrum. Although effective methods for harnessing this potential are known, the development is limited by two problems. Firstly there is the possibility of a reaction between the dye coated silver particles and the analytical reagents they label or other reagents and, secondly, to obtain stable intense SERRS it is preferable to maintain and reproduce a specific degree of aggregation among the silver particles—a procedure which can easily be upset during the analysis.

It is amongst the objectives of the present invention to obviate and/or mitigate at least one of the aforementioned disadvantages.

Generally speaking, the present invention is based on observations by the present inventors that a SERRS active dye can be encapsulated within a polymer coating, conferring stability to the dye, protecting the SERRS active dye from the environment and allowing more than one dye to be encapsulated such that more than one SERRS signal can be detected.

Thus, in a first aspect the present invention provides a SERRS active bead for use in identification of a target molecule, wherein the SERRS active bead comprises aggregated metal colloid and at least one SERRS active dye, encapsulated within a polymer shell.

SERRS refers to surface enhanced resonance Raman scattering. SERRS has been previously described, see for example Rodger, C. H., Smith, W. E., Dent, G., Edmonson, M., J. Chem. Soc. Dalton Trans., 1996, 5, 791 and references therein to which the reader is directed for background information. Surprisingly, there is a widespread fluorescence quenching mechanism on the silver surface. This means that almost all dyes give SERRS enabling the use of more extensive and simpler derivatisation chemistry than is possible by fluorescence. Further the specific enhancement of the dye provided by SERRS active particles is more than sufficient to allow the identification of the dye from the background signals normally observed from biological and industrial samples containing the target analyte. This eliminates the need for complex time consuming separation procedures required with many techniques before the analyte can be successfully identified.

It is to be appreciated that the term SERRS as used herein should not be construed as limiting and the present invention may also be applied to surface enhanced Raman scuttering (SERS), but SERRS is preferred.

Surprisingly, it has been found that encapsulating the SERRS active, metal nanoparticle and SERRS active dye within a polymer coating, does not prevent the SERRS signal from being easily detected. Moreover, the SERRS signal can be detected at a distance, in certain circumstances, of up to 10 metres from the beads.

The polymer shell may be formed from a wide variety of monomers and cross linkers including monomers that polymerise via chain-growth mechanisms and step-growth mechanisms capable of forming a bead which encapsulates the aggregated colloid and SERRS active dye. It is understood that a portion of the aggregated colloid may also be attached and/or embedded on the surface of the bead(s). The polymer shell may comprise more than one layer. For example beads may be formed which comprise some aggregated colloid on the surface. It is possible to then pass such beads back through the bead making process, with adding any metal colloid. In this manner an additional polymer cover or layer is created which contains no metal colloid particles on the surface of the bead. Nevertheless, in certain instances it may be desirable to have metal on the surface of the bead and this may be achieved using the metal colloid or by metal coating or lithographically depositing metal on the bead surface. An advantage of beads also including metal on their surface is that SERRS sensing can also be carried out on the surface of the bead which is additionally internally labelled.

Preferred classes of monomers and specific monomers include, but are not limited to, the following classes and derivatives thereof: acrylic acid and derivatives (e.g., 2-bromoacrylic acid, acryloyl chloride, N-acryloyl tyrosine, N-acryoyl pyrrolidinone), acrylates (e.g., alkyl acrylates, allyl acrylates, hydroxypropyl acrylate), methacrylic acid and derivatives (e.g., itaconic acid, 2-(trifluoromethyl) propenoic acid), methacrylates (e.g., methyl methacrylate, hydroxyethyl methacrylate, 3-sulfopropyl methacrylate sodium salt), styrenes (e.g., (2, 3 and 4)-aminostyrene, Styrene-4-sulfonic acid, 3-nitrostyrene), vinyls (e.g., vinyl chloroformate, 4-vinylbenzoic acid, 4-vinylbenzaldehyde, vinyl imidazole, 4-vinylphenol, 4-vinylamine, acrolein), vinylpyridines (e.g., (2,3, and 4)-vinylpyridine, 3-butene 1,2-diol), boronic acids (e.g., 4-vinylboronic acid), sulfonic acids (e.g., 4-vinylsulfonic acid), metal chelators (e.g., styrene iminodiacetic acid), acrylamides and derivatives (e.g., N-methyl acrylamide), methacrylamides and derivatives (e.g., N,N-dimethyl acrylamide, N-(3-aminopropyl)methacrylamide), alkenes (e.g., 4-pentenoic acid, 3-chloro-1-phenyl-1-propene) (meth) acrylic acid anhydride and derivatives (e.g., methacrylic anhydride), silicon-containing monomers (e.g., (3-methacryloxypropyl) trimethoxy silane, tetramethyldisiloxane), polyenes (e.g., isoprene, 3-hydroxy-3,7,11-trimethyl-1,6,10-dodecatriene), azides (e.g., 4-azido-2,3,5,6-tetrafluorobenzoic acid), thiols (e.g., allyl mercaptan). Acrylate terminated or otherwise unsaturated urethanes, carbonates and epoxies can also be used in this present invention, as can silicon-based monomers. Crosslinking agents that lend rigidity to the subject polymeric compounds are known to those skilled in the art, and include, but are not limited to, di-, tri-, tetra- and penta-functional acrylates, methacrylates, acrylamides, vinyls, allyls, and styrenes.

Silver, gold or copper-colloid surfaces, especially silver, are particularly preferred for use in the present invention. However, metal alloys can also be used.

The surface may be a naked metal or may comprise a metal oxide layer on a metal surface. It may include an organic coating such as of citrate or of a suitable polymer, such as polylysine or polyphenol, to increase its sorptive capacity. It can include dyes which covalently link onto the polymer.

The colloid particles are preferably aggregated in a controlled manner so as to be of a uniform and desired size and shape and as stable as possible against self-aggregation. Processes for preparing such unaggregated colloids are already known. They involve, for instance, the reduction of a metal salt (eg. silver nitrate) with a reducing agent such as citrate, to form a stable microcrystalline suspension (see P. C. Lee & D. Meisel, J. Phys, Chem. (1982), 86, p. 3.391). This “stock” suspension is then aggregated immediately prior to use. Suitable aggregating agents include acids (eg. HNO₃ or ascorbic acid), polyamines (eg. polylysine, spermine, spermidine, 1,4-diaminopiperazine, diethylenetriamine, aminoethyl)-1,3-propanediamine, triethylenetetramine and tetraethylenepentamine) and inorganic activating ions such as Cl⁻, I⁻, Na⁺ or Mg²⁺. To increase control over the process, all equipment used should be scrupulously clean, and reagents should be of a high grade.

The colloid particles can be of any size so long as they give rise to a SERRS effect—generally they will be about 4-50 nm in cross-section, preferably 25-36 nm, though this will depend on the type of metal.

The beads of the present invention are extremely small, typically less than 1 μm in diameter. Preferably the beads are about 10 nm to 1 mm in cross-section, more preferably about 10 nm to 1 μm in cross section, but obviously not smaller than the dye coated colloid particles which they encapsulate.

The beads of the present invention may contain a number of SERRS active nanoparticles (comprising of aggregated metal colloid and adsorbed SERRS active dye or dyes) each of which may be identically labelled or differently labelled in order to increase the multiplexing capability. That is, a single bead may comprise of one or more SERRS active dyes. If more than one dye is incorporated within a bead, the SERRS signal obtained, will be a combination of the SERRS signals obtained from the various dyes used and by varying the dyes and/or relative levels of dyes within a bead, a “library” of beads may be formed, with discernable SERRS signals.

The action of the silver nanoparticles with the polymer in the formation of the beads is to reduce the size of the beads. This provides additional control over the bead chemistry and means that a large range of sizes can be formed. Thus, once the polymer is formed the SERRS bead arrangement is locked in position so that the state of aggregation chosen is preserved. Further, the polymer protects the silver nanoparticles from the environment.

The polymer used in these beads may further comprise functional groups (e.g. carboxylic acids, active esters of carboxylic acids, alcohols, amines, epoxides, chloromethyl styryl groups, vinyl groups, thiol groups, maleimide and succinimide) or may be further derivitised by a linking group or groups, such that target molecules may react and become bonded to the surface of the particles. The advantage of these groups is that they can act as points to covalently or non-covalently attach a molecule to provide a specific capture or analysis. The term linking group refers to any group which facilitates adherence between one or more target molecules and one or more SERRS active particles. The adherence may comprise any sort of bond, including but not limited to covalent, ionic, hydrogen bonding, van der Waals forces, or mechanical bonding.

In a further aspect, the present invention provides a method of preparing SERRS active beads comprising the steps of:
1) forming an aggregated metal colloid comprising a SERRS active dye absorbed thereto;
2) mixing the aggregated metal colloid with a monomer in a suitable solvent; and
3) effecting polymerisation of the monomer so as to form polymer beads encapsulating the aggregated metal colloid.

Alternatively beads may first be made without aggregated colloid comprising a SERRS active dye absorbed thereto. The beads may then be heated (e.g. to 80° C.) in order to render them porous and capable of taking of said aggregated colloid comprising a SERRS active dye absorbed thereto. Cooling the beads traps the colloid particles within the bead. Dye/colloid can be removed by raising the temperature and steeping the beads in a suitable buffer.

The polymer beads may be further reacted so as to add functional or linking groups to an outer surface of the bead thereby allowing a molecule to be attached to the bead.

Examples of suitable target molecules include, nucleic acids, proteins such as antibodies, aldehydes, thiols, amines, explosives, drugs of abuse, therapeutic agents, metabolites and environmental pollutants. However, the beads, due to their extremely small size may also find application alone, e.g. as a security marker. The beads may therefore be applied to a surface by being contained within for example an aerosol spray, paint, ink or the like. The beads would generally be invisible to the naked eye, but would be loaded with a particular SERRS active dye or dye combination such that a specific SERRS signal could be detected. This may be particularly applicable to prevent/minimise counterfeiting.

Attaching suitable species such as DNA sequences to the polymer surface carries out the analysis/labelling procedure. In this way, the analysis process and the SERRS process are separate and both can be optimised.

The beads of the present invention could have broader applicability as a labelling technology. They could be used for example both for protein and antibody detection. In the case of proteins, the simplest example would be to attach one of a pair of proteins to the SERRS active surface and attach a fluorescent label to the other protein. When the pair complex, the bead would show both fluorescence and Raman scattering and when the pair separate, the Raman spectra would be detected only on the bead. A spectrometer exists which could be adapted to provide both fluorescence and Raman scattering simultaneously.

Moreover, the beads could be further adapted to for example, assist their separation when in use. The beads may be attached to a surface specifically by derivatising the surface of the beads with, for example, a DNA molecule which recognises a target and contains a biotin group, and could be brought down on a streptavidin surface. Alternatively/additionally, the beads could for example be magnetised by the incorporation of magnetic species within and/or on the surface of the beads weighted by the addition of materials which would allow the particles to be separated on the basis of their weight. The magnetic beads could then be brought down by using a small magnet to, for example, concentrate the particles in the beam of the spectrometer.

Suitable magnetic species may be in the form of magnetic nanoparticles which are incorporated into the bead. Such magnetic nanoparticles may include any ferromagnetic material in nanoparticle form including iron, magnetite, ferrites and compounds of other metals such as nickel and cobalt.

Incorporation of the magnetic nanoparticles may be achieved by combining a solution comprising said magnetic nanoparticles with a colloid/dye mixture and thereafter forming the SERRS active bead. The formed beads may thereafter be functionalised, as previously described.

In the case of antibodies, a very simple assay could be developed which eliminates some of the uncertainties with competitive and displacement assays in that, an antigen labelled with a fluorophore could be added to an antibody immobilised on the SERRS active bead. When the beads are removed from solution by centrifugation or magnetic fields, those beads with fluorophores can be interrogated and the Raman code read to determine which labels had been displaced. Alternatively, the beads could be used as specific labels in a sandwich assay in which the antigen is trapped on an antibody on a surface such as a plate or magnetic bead and A SERRS labelled antibody coated particle will adhere to the surface by attaching to the trapped antigen. The antigen may also-initially be trapped by the antibody on the labelled particle. Protein recognition may be probed similarly by attaching a bead to each of the two proteins or by attaching a bead to one protein and another label such as a fluorophore to the other.

In yet another embodiment of the invention, molecular imprinting methods can be applied to impart specific molecular recognition properties to the polymer shell via templating approaches (see for example Wo 00/41723). This generic approach can in principle be applied to impart affinity to the polymer shell for a range of analytes, including analytes of clinical, dietary, forensic and environmental relevance.

The method for obtaining the SERRS spectrum, may be conventional. However, the following might apply to the spectroscopic measurements:

Typically, the methods of the invention will be carried out using incident light from a laser, having a frequency in the visible spectrum ie. ⁻380 nm-780 nm, particularly between 400 nm-650 nm (the exact frequency chosen will generally depend on the dye used in each case—frequencies in the red area of the visible spectrum tend, on the whole, to give rise to better surface enhancement effects). However, it is possible to envisage situations in which other frequencies, for instance in the ultraviolet (ie. 200 nm-400 nm) or the near-infrared ranges (700 nm-1100 nm), might be used. Thus, SERRS detection may be conducted between about 200 nm-1100 nm.

The selection and, if necessary, tuning of an appropriate light source, with an appropriate frequency and power, will be well within the capabilities of one of ordinary skill in the art, particularly on referring to the available SERRS literature. To achieve highly sensitive detection, using SERRS, a coherent light source is needed with a frequency at or close to the absorption maximum for the dye. If lower sensitivities are required, the light source need not be coherent or of high intensity and so lamps may be used in combination with a monochromator grating or prism to select an appropriate excitation frequency.

Several devices are suitable for collecting SERRS signals, including wavelength selective mirrors, holographic optical elements for scattered light detection and fibre-optic waveguides. The intensity of a SERRS signal can be measured for example using a charge coupled device (CCD), a silicon photodiode, or photomultiplier tubes arranged either singly or in series for cascade amplification of the signal. Photon counting electronics can be used for sensitive detection. The choice of detector will largely depend on the sensitivity of detection required to carry out a particular assay.

Note that the methods of the invention may involve either obtaining a full SERRS spectrum across a range of wavelengths, or selecting a peak and scanning only at the wavelength of that peak (ie. Raman “imaging”). It is also possible to detect all Raman scattering using only a filter to remove reflected light, Raleigh scattering etc and a detector such as photodiode.

Apparatus for obtaining and/or analysing a SERRS spectrum will almost certainly include some form of data processor such as a computer.

Raman signals consist of a series of discrete spectral lines of varying intensity. The frequencies and the relative intensities of the lines are specific to the SERRS active dye being detected and the Raman signal is therefore a “fingerprint” of the dye. If a SERRS analyser is being used selectively to detect a mixture of dyes then it will be necessary to detect the “fingerprint” spectrum for identification purposes.

Once the SERRS signal has been captured by an appropriate detector, its frequency and intensity data will typically be passed to a computer for analysis. Either the fingerprint Raman spectrum will be compared to reference spectra for identification of the detected Raman active compound or the signal intensity at the measured frequencies will be used to calculate the amount of Raman active compound detected.

In summary, the development of polymer particles containing SERRS active particles for multiple labelling of analytes is described. The colloid particles are dye labelled, pre-aggregated and locked into the polymer so that the SERRS effect is switched on permanently and the analytical event is separately carried out by, for example, covalently linking a molecule to be detected to the polymer surface. The main advantages are that the particles give very intense, stable SERRS. For simple, sensitive identification of a specific particle, there is a huge multiplex labelling capability in suspension which is unrivalled by any other technique and the SERRS active nanoaggregates are protected from the environment. In turn, this enables identification and quantitation of specific target analytes such as DNA sequences or peptides in-situ at ultra-low concentrations in a complex mixture.

In a further aspect the present invention provides a method of detecting a target molecule in a sample, the method comprising the steps of:
a) providing a SERRS active bead(s) according to the present invention wherein the bead has been generated so as to have a specifically identifiable SERRS signal and the surface of which has been modified so as to have attached thereto a moiety capable of binding said target molecule;
b) contacting said bead(s) with the sample so as to allow any of said target molecule in the sample to bind to said bead(s); and
c) detecting said target molecule by way of detecting the specifically identifiable SERRS signal obtained from a bead or beads being bound to said target molecule.

Typically the target molecule may be an antibody or antigen, in which case the SERRS active bead is modified so as to have a corresponding antigen or antibody respectively, attached to the surface of the bead. A conventional sandwich assay could be employed where a first antibody is attached to a surface, for binding to a specific protein which may be in a sample. The bead may be derivatised to include a second antibody which is also specific for said protein. If the protein is present in a sample it effectively becomes sandwiched between the two antibodies and this can be detected by SERRS being carried out on the bead attached to the second antibody. Other molecules which associate naturally may also be detected such as a receptor/ligand complex, with one of said complex immobilised on the bead surface.

In a preferred embodiment the method is used to detect a particular nucleic acid sequence in a sample. In this case, the bead may be modified so as to have a molecule capable of specifically hybridising to a nucleic acid molecule which comprises the particular nucleic acid sequence, attached to the surface of the bead. The molecule capable of specifically hybridising may be of a sufficient length to enable a simple hybridisation and subsequent detection to occur. Alternatively the molecule capable of specifically hybridising to the target nucleic acid molecule may be a relatively short molecule, say less than 50 nucleotides, e.g. less than 25 nucleotides in length, designed to act as a primer to allow hybridisation to said nucleic acid molecule and chain extension therefrom. In this manner the molecule attached to the surface of the bead will generally be attached via its 5′ end so that the 3′ end is capable of binding and initiating chain extension. Typically a further primer designed to bind to the target nucleic acid molecule, at a distance, e.g. 100 nucleotides to 2000 nucleotides, from the bead bound primer, may also be utilised. This can allow generation of a double stranded molecule, which incorporates the target nucleic acid sequence, using an appropriate DNA polymerase and necessary dNTPs (deoxynucleotide tri-phosphates), well known to those skilled in the art.

Indeed an amplification reaction e.g. PCR can be carried out in order to effectively enhance the amount of target nucleic acid to be detected. The further primer may also be modified by suitable means so as to comprise a moiety which is capable of binding to a particular surface. For example the primer may comprise a biotin molecule at the 5′-end of the primer so as to enable any amplified product to be capable of binding to a surface coated with streptavidin. In this manner an amplified product can be generated which can be bound to a surface via one end of the amplified product, whilst the free end incorporates a SERRS active bead which can easily be detected.

Due to the ability of being able to generate SERRS active beads with specific different SERRS signals it is possible to conduct what is known in the art as a multiplex reaction using a number of differently labelled beads each with different immobilised binding moieties e.g. oligonucleotides. In the case of oligonucleotides, each one may be designed to identify, for example, a particular polymorphism in a nucleic acid molecule, by incorporating specific sequences at the 3′ end which will only allow chain extension to occur when complementary binding at the 3′ end occurs. That is, when the nucleotide at the 3′ end does not hybridise to the target, chain extension cannot occur therefrom. By carrying out an amplification reaction using another primer designed to generate an amplified product, it is possible to easily detect which polymorphism is present in a target nucleic acid molecule.

If an amplification reaction is carried out, a dye, such as Sybr Green can be used as an initial indicator that an amplified product has been obtained. This product can thereafter be identified by carrying out SERRS detection. This optional step can be employed such that only reactions which are identified as generating an amplified product are thereafter analysed by SERRS detection.

In summary the key advantages of the present invention are,

1) Sensitivity: Each bead is a separate sensing unit capable of housing up to millions of SERRS active dye labels. The entire available silver surface can be closely packed with dye labels. The intra molecular quenching mechanisms that prevent close proximity of two labels with, for example, fluorescence, do not apply in SERRS. Thus for one bead, the signal is very strong and easily and quickly detected at low concentration.

2) Robustness: The process of aggregation, essential to obtain the maximum signal from SERRS, is carried out during formation of the bead. Thus, it is preset and protected from the environment by the polymer making the signal long-lived, constant and unaffected by any biological event. In addition, the sensing species used to find a specific analyte is covalently attached to the polymer surface.

3) Multiple detection: The beads may be manufactured so as to contain many codes “written” by using different dye mixtures in each of a number of colloidal suspensions and by adding aggregated colloid and dye from more than one suspension to a specific bead. The sharp nature of the SERRS peaks then enables recognition of each bead type in a mixture of beads without any separation. Since all chromophores that adhere strongly to the SERRS active surfaces can be used, much simpler and more extensive dye chemistry is possible than with fluorescence. For example, the pattern of SERRS signals from a dye such as an azo is very sensitive to substitution round the chromophore making it much easier to create multiple labels than is the case with fluorescence. This greatly increases the coding potential with relatively simple dyes.

Moreover, because the beads include a metal, a significant mass is provided to the bead. This weight may also be used in detection and/or separation techniques employing the beads. For example, a DNA strip recognised as in FIG. 2 could be trapped on the surface of a streptavidin coated plate of a quartz microbalance balance and the mass change detected. The heavy nature of the bead would make this mass change simple to detect increasing the sensitivity of the quartz balance compared to conventional trapping of the DNA strip. After detecting the weight change, the specific particle causing it could be detected by a weight change.

The present invention will now be further described by way of example and with reference to the Figures which show:

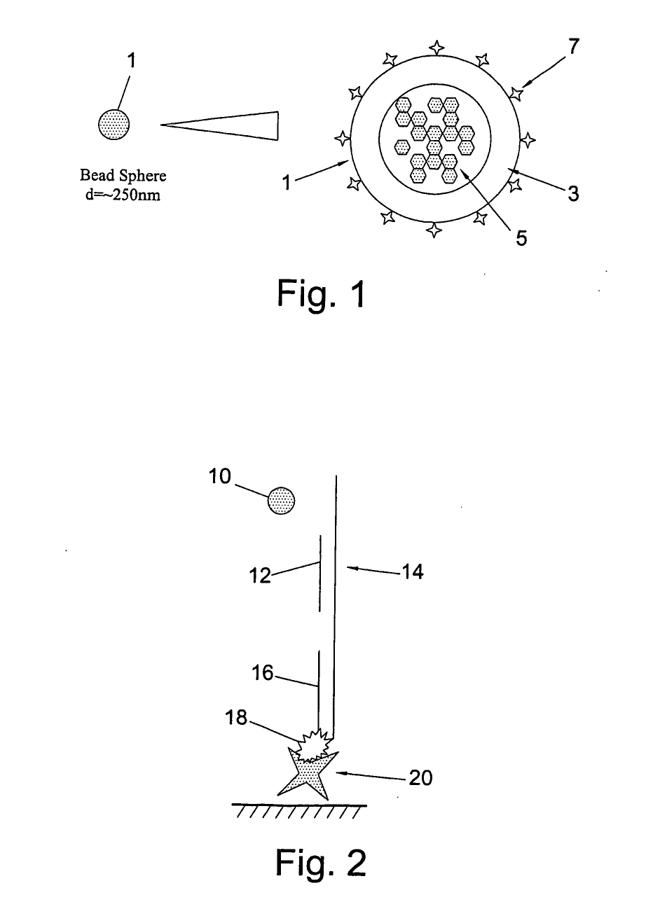

FIG. 1 shows a schematic representation of a SERRS active bead according to the present invention;

FIG. 2 shows in schematic a potential use of a SERRS active bead according to the present invention;

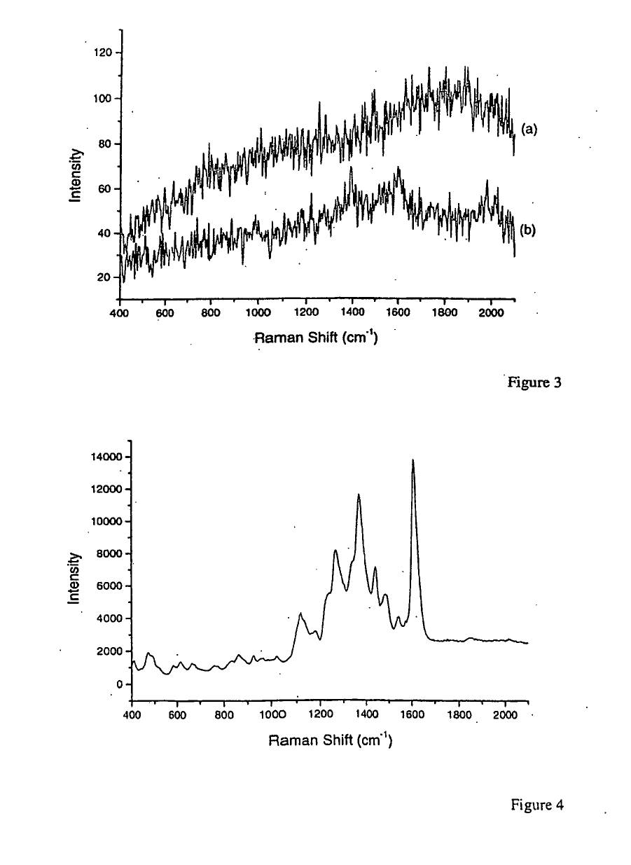

FIG. 3 shows the Raman spectra of control beads and the SERRS of blank beads in Example 2;

FIG. 4 shows the SERRS of a dye in a dye coated bead sample;

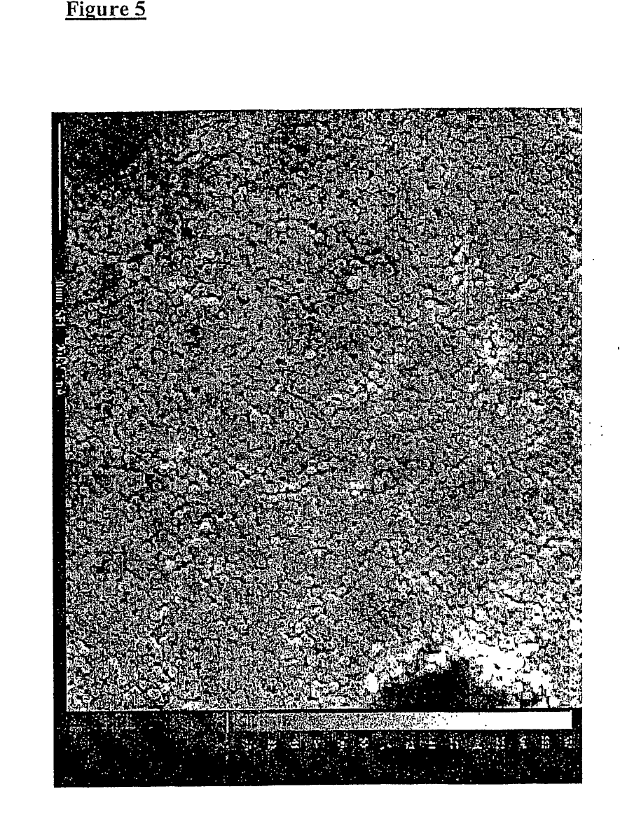

FIG. 5 is a SEM image of dye coated beads, the measurement bar is 10 μm in length and the average diameter of the beads shown in ˜0.8 μm;

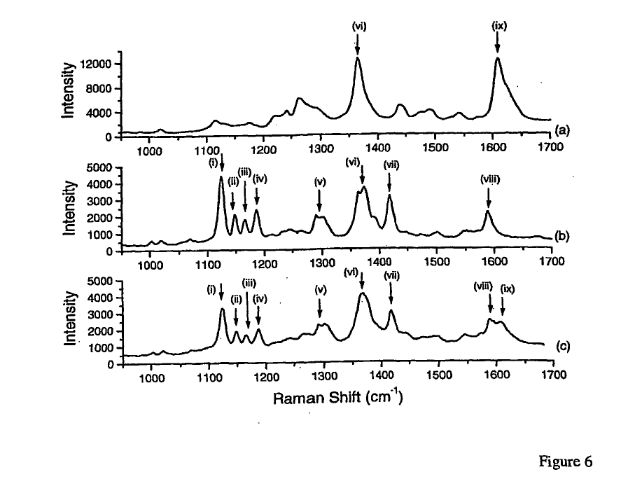

FIG. 6 shows the SERRS spectra of ABT DMOPA, 8HQPK and a 1:10 ratio mixture of both dyes;

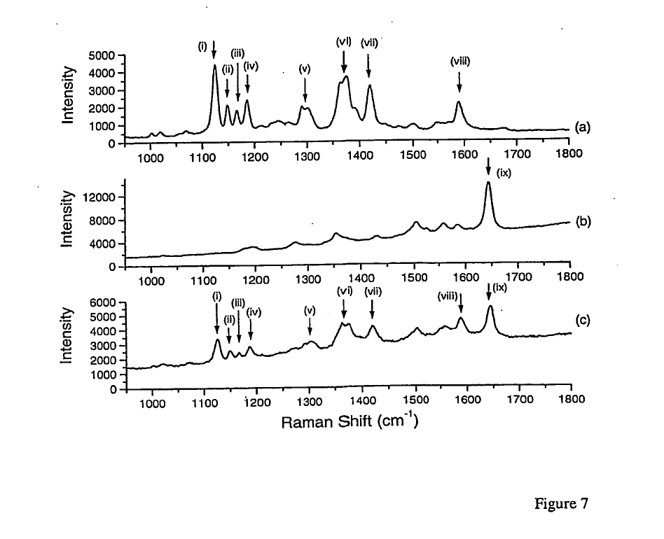

FIG. 7 shows the SERRS spectra 8HQPK, benzotriazole rhodamine B and a 3:1 mixture of both dyes; and

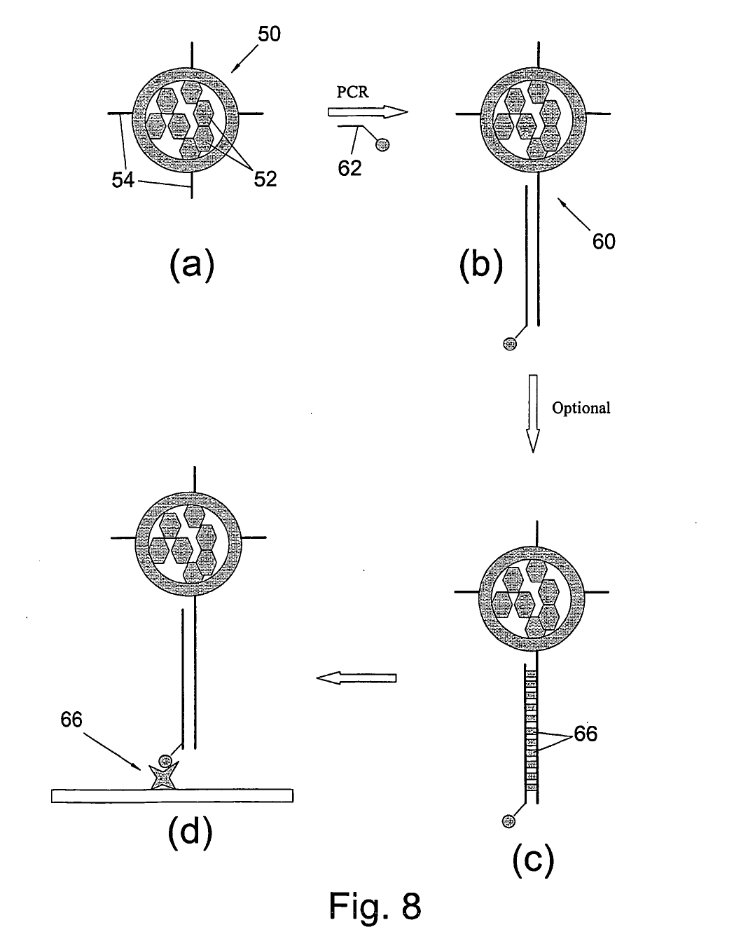

FIG. 8 shows a schematic representation of how a SERRS active bead according to the present invention may be used to identify a specific nucleic acid sequence, using an amplification reaction.

### EXAMPLE SECTION

### Example 1

Colloid Preparation

Citrate-reduced colloid was prepared according to the method of Lee and Meisel (Rodger, C., Smith, W. E., Dent, G., Edmonson, M., J. Chem. Soc, Dalton Trans., 1996,5,716; Munro, C. H., Smith, W. E., Garner, M., Clarkson, J. and White, P. C., Langmuir, 1995, 11, 3712). All glassware was throughly cleaned using Aqua Regia (3:1, HCl:HNO₃) and then thoroughly rinsed with water followed by distilled water. Silver nitrate (90 mg) was dissolved in distilled water (500 ml) at a temperature of 40° C. The solution was then quickly heated using a Bunsen burner to a temperature of 98° C. At this point 10 ml of a 1% solution of tri-sodium citrate was added. The temperature was then carefully controlled, keeping the temperature constant at 98° C. for 90 minutes. After this period of constant temperature the colloidal suspension was cooled and its UV absorbance spectrum measured. Colloid with a maximum UV absorbance within the range 403-410 nm was used in the experiments detailed below.

The silver pellets used in the polymerisations were prepared by centrifuging 12 tubes containing 12 ml of citrate-reduced colloid (λₘₐₓ of 406 nm) at 3000 r.p.m. for 20 minutes. The supernatant from each tube was removed and the remaining silver was concentrated into one tube. The concentrated pellet was then re-suspended in acetonitrile using ultrasonication, which was then centrifuged at 3000 r.p.m. for 40 minutes. The supernatant was removed once again, the resultant pellet re-suspended in acetonitrile using ultrasonication (5 ml) and the suspension incorporated into the polymerisation.

The dye-loaded samples were prepared in exactly the same way as above, but in the 12 tubes containing citrate-reduced colloid 150 μl of a 1×10⁻⁶ M solution of ABTDMOPA[4(5′-azobenzotriazoyl)-3,5-dimethoxyphenylamine] was added.

Bead Synthesis

In what follows the Control polymer contained no colloid whereas the Blank polymer contained colloid but no dye. The Dye-Loaded polymer contained colloid and dye.

Three polymerisations (Control, Blank and Dye-Loaded) were carried out in three separate 50 ml thick-walled glass tubes. The reaction vessel for the Control polymer was charged with acetonitrile (25 ml), divinylbenzene 80 (0.5 g) and azobisisobutyronitrile (AIBN) (0.005 g), the reaction vessel for the Blank polymer was charged with acetonitrile (20 ml), the dye-free suspension of colloid in acetonitrile (5 ml), divinylbenzene 80 (0.5 g) and AIBN (0.005 g), and the reaction vessel for the Dye-Loaded polymer was charged with acetonitrile (20 ml), the dye-loaded suspension of colloid in acetonitrile (5 ml), divinylbenzene 80 (0.5 g) and AIBN (0.005 g). The reaction vessels were shaken to dissolve the monomer and the AIBN, cooled on an ice-water bath for ⁻10 minutes and then sparged with oxygen-free nitrogen gas to remove dissolved oxygen. The tubes were then sealed and polymerisation effected by heating at 60° C. on a low-profile roller for a period of 24 hours. Following polymerisation the polymer beads were isolated by filtration on membrane filter (0.2 μm), washed with several volumes of fresh acetonitrile and then dried in vacuo at 40° C.

Bead Modification

1-Ethyl-3-(3-dimethylaminopropyl) carbodiimide (EDC) (500 mg) in 25 ml of 0.1 M MES buffer (pH 4.5) was added to the polymer beads and incubated at 25° C. for two hours to form the amine surface active species. FIG. 1 shows a schematic of a bead (1) prepared as described above. The bead (1) is formed of polymer shell (3) encapsulating aggregated colloid and SERRS active dye beads (5). The surface of the polymer shell (3) has been functionalised so as to have amino surface active species (7) which are capable of reacting with DNA. 3 μg of oligo per gram of bead in 25 ml of sodium phosphate buffer (pH 7.2) were added to the activated beads and reacted at 25° C. overnight. The beads were removed from solution by centrifugation and re-suspended in phosphate buffer (pH 7.2) ready for use.

In a simple example (see FIG. 2) of the use of the beads (1) prepared as described above, the oligonucleotide (12) is designed to recognise a specific sequence of DNA. When the target sequence (14) is present in a DNA extract, it will hybridise to the oligonucleotide (12) and thus be captured by the bead (10). A second oligonucleotide (16) containing for example a biotin group (18) is also hybridised and the system brought out on a streptavidin plate or bead (20). This assay specifically recognises the target sequence (14), which hybridised both the particle (10), and the biotinylated sequence (16). Alternatively, if magnetic particles are added to the bead, it and the hybridised target could be brought down on a suitable surface without the biotin step. In both methods various approaches can be used to remove the unincorporated probe. In the first method, no label is brought down if extra biotin is added to the streptavidin plate and consequently it is an interferent that does not need to be rigorously removed. However, the size of the complex created with the label makes separation easy and in either method removal of an unincorporated probe can be achieved using a flow cell system or a short chromatography step. Alternatively magnetic separation can be achieved by using magnetically coated streptavidin beads to pull the complex from solution.

### Example 2

A further set of silver pellets were then prepared using 12 centrifuge tubes. Blank beads contained 9.6 ml colloid and 2.4 ml water per tube. Dye coated beads contained 2.4 ml dye (1×10⁻⁶ M) and 9.6 ml colloid per tube.

All tubes were left overnight after adding the dye and centrifuged for 40 minutes. Pellets were re-suspended in 10 ml acetonitrile and re-centrifuged for a further 40 minutes (no ultrasonication).

Pellets were then re-suspended in a further 5 ml of acetonitrile and ultrasonicated for 2 minutes before addition to the polymer reaction mixture.

0.5 g divinylbenzene and 0.005 g AIBN were then dissolved in a total volume of 25 ml acetonitrile (including the 5 ml of colloid). Samples were degassed with N₂ for 10 minutes. The samples were then placed on rollers at 37° C., heated to 60° C. and left to polymerise for 24 hours.

The samples were then filtered through a 0.2 mm pore size filter paper, washed with acetonitrile and dried in a vacuum oven.

A small amount of the beads (i.e. control, blank and dye containing beads) were then suspended in ⁻1 ml of acetonitrile, ultrasonicated, dropped onto a microscope slide and allowed to dry. The spectra shown in FIGS. 3 and 4 were then recorded on the system 100 Renishaw microprobe Raman spectrometer, x20 objective, 100% power ⁻8 mW) ls, la.

FIG. 3 shows the Raman of control beads which are identified as (a) and the SERRS of blank beads which are identified as (b)

FIG. 4 shows the SERRS of the dye in the dye containing bead sample.

SEM Images

A small amount of the beads were then suspended in a solution of PVA using ultrasonication. The beads were then dried into a small glass disc, dried and stored under vaccum before analysis. The SEM image in FIG. 5 was then recorded on the Cameca SX100 Electron Microprobe Microanalyser using 5 kV electron beam. The large bead is ⁻1.5 μm and the small beads are ⁻0.5 μm.

Dye Mixtures in PVA

FIGS. 6 and 7 are Raman spectra of two examples of two mixtures in the PVA stable substrates.

FIG. 6 shows the SERRS spectra of (a) ABT DMOPA (GM19), (b) 8HQPK, and (C) a 1:10 ratio mixture of both dyes. The spectra were collected on the Renishaw microscope system, 514, 5s, la, 100% power (approx. 3 mW), X5 objective.

In FIG. 6, the main peaks of each of the two dyes (a) ABT DMOPA and (b) 8HQPK can be clearly identified in the spectrum of the mixture (c). Peaks labelled (i-v), (vii) and (viii) all originate from the 8HQPK dye (i.e. 1-[4-(8-hydroxy-quinolin-5ylazo)-phenyl]-ethanone) while the peak labelled (ix) is from the ABT DMOPA dye. The peak at ⁻1360 cm⁻¹ in the mixture spectra (c) is a combination of the peaks at that position from both dyes. The intensity of this peak has increased relative to the other peaks in the spectrum.

FIG. 7 shows the SERRS spectra of (a) 8HQPK, (b) Benzotriazole rhodamine B and (c) a 3:1 ratio mixture of both dyes. Collected on the Renishaw microscope system, 514 nm, 5s, 3a, 100% power (approx. 3 mW), x5 objective. In FIG. 7, the main peaks of each of the two dyes (a) 8HQPK and (b) BTRB can be clearly identified in the spectrum of the mixture (c). Peaks labelled (i-viii) all originate from the 8HQPK dye (i.e. Benzotriazole Rhodamine B-{9-[4-(Benzotriazol-5-yl sulfamoyl)-2-methyl-phenyl]-6-diethylamino-xanthen-3ylidene}diethyl-amonium) while the peak labelled (ix) is from the BTRB dye.

### Example 3

### Use of Magnetic Beads

Bead Preparation

50 ml of a solution of magnetic nanoparticles was added to 50 ml of silver colloid treated with the dye mixture as in Example 1. The mixture of nanoparticles was then centrifuged and the bead process carried out as in Example 1. The bead was then functionalised as in Example 1 with oligonucleotides.

Method (a)

This method combines separation and purification using the magnetic properties of the particle.

Using a sample of the PCR product intended for the assay in example 2 where a biotin strip and a bead have been hybridised to the target DNA, a small flat tip from an electro magnet was introduced into a micro titre plate well containing PCR product. The magnet was switched on and all magnetic beads collected. The magnet was then removed from the solution, dipped into a second well to wash it and then into a third well with a streptavidin coated area as in example 2. The electro magnet was switched off in the third well and the beads released. Following a period of two minutes in which the beads were allowed to attach to the streptavidin area, the magnet was switched on again. This removed all unattached beads.

In this way, all unincorporated biotin primer remained in the first well and all beads not attached to the target strip were removed in the first step and biotin labelled DNA not attached to the target strip was removed in the second step. The beads remaining in the foot of the well after the second step were interrogated as in Example 2 and the SERRS code used to identify the bead and hence the target DNA.

Method (b)

This method illustrates the selectivity advantage of the use of derivatised beads containing only magnetic particles with no labelled silver particles. Commercial derivatised beads could also be used.

A batch of beads were prepared as described in Example 1 but without the addition of the silver nanoparticles and derivatised using specific oligonucleotide sequences to recognise a specific target DNA. Also incorporated into the PCR mix were other beads with the silver labels incorporated into the bead and no magnetic particles. They contain a second oligonucleotide sequence designed to recognise a different region of the same target DNA. Following PCR, the electromagnet is added to the well and switched on. Only samples with magnetic beads are attracted to its tip. The tip was then interrogated under the Raman microprobe. Only tips containing labelled beads were observed. This method is effective because it concentrates all the beads in the microscope interrogation volume. This increases sensitivity. It is reliable and selective because it involves a double hybridisation event.

Method (c)

This method illustrates the combined use of Raman and fluorescence with magnetic beads.

Using the PCR mix from example 4 where Sybr green has been added to the DNA, the electromagnet was dipped into the PCR mix. It was switched on and the magnetic beads removed. The mix was then directly interrogated under the Raman microscope. A frequency was chosen so that the fluorescence and the Raman signals were detected on separate arts of the CCD detector. The method is effective because of the sensitivity created by collecting the beads in a small area for immediate detection. The fluorescence from Sybr green detects a hybridisation even within a conventional PCR machine so that a single well can be selected from an array for a fast one step identification analysis.

### Example 4

### Use of SERRS Beads for DNA Detection

In this example SERRS Beads can be used as a label for detection of a specific DNA sequence. The format is based around a PCR assay to demonstrate the high sensitivity of the SERRS Beads and also the increased capacity for multiplexing. An initial fluorescence screen can indicate the tubes or wells that have produced a response. These “hot” wells can be examined by SERRS and the actual DNA sequence or sequences that have hybridised determined.

Modified beads can be prepared according to Example 1 in which an outer surface of the bead has been modified so as to allow immobilisation of an oligonucleotide the 3′ end of which is directed away from the outer surface of the bead. This can easily be achieved by for example attaching a group to the 5′ end of the oligonucleotide during its synthesis and modifying the surface of the bead so that the group on the 5′ end adheres to the surface of the bead.

FIG. 8a shows a schematic representation of such a bead 50, which has a specific SERRS signature by virtue of the dye labelled colloid particles 52 found within the bead 50, with a number of identical oligonucleotides 54 adhered to the surface of the bead 50.

PCR Amplification of the Target

PCR is used to amplify a specific piece of double stranded DNA from the sample. This makes use of the existing specificity of PCR by using allele specific oligonulcleotides for genotyping. Initially, primer sequences can be chosen to detect the mutational status of the most common mutation found in a particular disease. To analyse the genotype of the sample DNA, two forward primers may be used along with one reverse primer in a multiplexed amplification refractory mutation system, ARMS. The forward primers are immobilised via a 5′-terminus linker, onto different SERRS Beads that act as the labels for the specific primer. The reverse primer is tagged at the 5′-terminus with biotin. As the PCR progresses the product will be built up on the SERRS Bead with biotin incorporated at the end of the amplicon away from the SERRS Bead. Alternatively, it is possible to carry out a conventional PCR of the target followed by introduction of the SERRS Beads when there is an issue with performing PCR on the SERRS Beads.

In the case of for example a pentaplex reaction five different primers can be immobilised on five different SERRS Beads and the PCR performed with all the beads mixed together with the sample.

FIG. 8b shows schematically a labelled product 60 which has been obtained following a PCR reaction. Amplification using the primer attached to a SERRS bead (see FIG. 8a) and a biotinylated primer 62 results in the generation of a biotinylated product which also incorporates a SERRS reactive bead.

Initial Screening of the PCR [Optional]

The success of the PCR can be measured by the increase in fluorescence of, for example, Sybr Green (64, see FIG. 8c) as the amount of double stranded DNA increases. Use of a dissociation curve at the end of the PCR will confirm the presence of PCR product and not primer dimer. Thus if there is any amplification then the fluorescence will show this. It is possible to use a QPCR instrument to measure the increase in fluorescence during the PCR however a conventional plate reader could also be used after the PCR to rapidly analyse the tubes. Control tubes of no template controls and Beads with nonsense DNA can be used a deemed appropriate.

Immobilisation of the SERRS Beads on a Surface

Once the tubes that have an increase in fluorescence had been optionally identified as having PCR product, the contents can be spotted onto a streptavidin coated slide (66, see FIG. 8d). This immobilises the biotinylated PCR products with the SERRS Beads on the surface of the slide. The biotinylated primers will also be immobilised (but will not have a specifically detectable SERRS signal) but everything else can be washed away in a series of washes. (This will include the intercalated Sybr Green which could fluoresce and interfere with the SERRS signals). Thus, as series of SERRS Beads will now be immobilised on the surface of the slide ready for interrogation by SERRS.

SERRS Analysis of the Immobilised Beads

SERRS spectra can be taken on the immobilised SERRS Beads to identify the actual primer sequences that have been used to generate the PCR products. The ability of SERRS to identify the components of a mixture without separation can be used to identify the SERRS Beads from particular tubes with a multiplex e.g. pentaplex likely to be possible by eye although statistics can also be used to confirm this for the non-specialist with a view to increasing the degree of multiplexing.

Typically the whole setup should take little more than the time for a standard QPCR experiment i.e. about 150 mins if a dissociation curve is used or about 60 mins if less cycles are used in a standard PCR cycler. The PCR setup can be arranged in such a way that the effective concentration of the primers on the beads and the final volume will be entered into a spread sheet and all other values automatically displayed. This makes setting up the PCR very simple and reduced systematic error in the calculations. After the PCR has finished there may be a need to pipette the contents of the strip tubes into microtitre plates for either plate reading or to be compatible with robotic handling. Alternatively direct pipetting onto the streptavidin slide is an option.

It may be possible to use linear extension as opposed to amplification to incorporate the SERRS Bead into a long double stranded piece of DNA. Fluorescence detection is unlikely to pick this up but the SERRS Beads will report on the enzymatic extension whether there is one piece of DNA or millions.

### Example 5

### SERRS Bead DNA Binding Assay, Exploiting the Biotin—Streptavidin Binding Affinity

An example of the SERRS beads binding assay is presented here. In this example the 110 base long sequence of a mutant Cystic Fibrosis gene is analysed.

The active surface of a SERRS bead, labelled with an azo dye, was derivatised with a short oligonucleotide probe, complementary to a sequence of DNA found on a mutant Cystic fibrosis gene. The mutant CF strand was linked to a biotin group at the opposite end of the sequence to where the complementary probe was synthesised. The SERRS-DNA probe was hybridised to the CF-biotin sequence and immobilised on a streptavidin plate.

Method

The SERRS beads were synthesised using the method outlined earlier with divinyl benzene 80 and methacrylic acid. The surface of the beads is covered with a number of carboxylic acid groups. These active acid groups are used for attaching oligonucleotides and other molecules to the surface of the bead.

DNA Sequences

Two sequences were synthesised using the Expedite DNA Synthesiser

Sequence A containing an amine linker, X.
Sequence A -X-GTATCTATATTCATCATAGGAAACACCATT
Sequence B containing biotin, B
Sequence B -B-GACTTCACTTCTAATGATGATTATGGGAGA

(X=aminohexamethylene and is added to the 5′-terminus to produce an oligonucleotide with a primary amine group at the end of a carbon chain after solid phase synthesis and purification).

Sequence A (400 μl, 0.6 mg/ml) was purified and coupled to SERRS active beads (70 mg) using 1-ethyl-3-(3-dimethylaminopropyl) carbodiimide (EDC, 37 mg) and N-hydroxysuccinimide (24 mg) in 0.1M PBS (750 μl) buffer and acetonitrile (100 μl).

Sequence B (0.5 μM) was used in the PCR amplification reaction with its CF complement and a reverse primer. The product was purified and added to the oligo derivatised beads with additional Sequence B and heated to 95° C. for 10 mins.

After this 100 μl of the dispersed solution was dotted onto a streptavidin plate and left to stand for 3 hrs in a humid chamber. The DNA derivatised beads were used as a control. The plate was washed with 1% SDS solution, water and acetonitrile. The beads were dried and examined by Raman photospectroscopy.

1. A SERRS active bead for use in identification of a target molecule, wherein the SERRS active bead comprises aggregated metal colloid and at least one SERRS active dye, encapsulated within a polymer shell.

2. A SERRS active bead according to claim 1 wherein the polymer shell is formed from a wide variety of monomers and cross linkers including monomers that polymerise via chain-growth mechanisms and step-growth mechanisms capable of forming a bead which encapsulates the aggregated colloid and SERRS active dye.

3. A SERRS active bead according to claim 1 wherein said polymer shell or shells is/are formed from any of the following classes and derivatives thereof: acrylic acid and derivatives (e.g., 2-bromoacrylic acid, acryloyl chloride, N-acryloyl tyrosine, N-acryoyl pyrrolidinone), acrylates (e.g., alkyl acrylates, allyl acrylates, hydroxyypropyl acrylate), methacrylic acid and derivatives (e.g., itaconic acid, 2-(trifluoromethyl) propenoic acid), methacrylates (e.g., methyl methacrylate, hydroxyethyl methacrylate, 3-sulfopropyl methacrylate sodium salt), styrenes (e.g., (2,3 and 4)-aminostyrene, styrene-4-sulfonic acid, 3-nitrostyrene), vinyls (e.g., vinyl chloroformate, 4-vinylbenzoic acid, 4-vinylbenzaldehyde, vinyl imidazole, 4-vinylphenol, 4-vinylamine, acrolein), vinylpyridines (e.g., (2,3 and 4)-vinylpyridine, 3-butene 1,2-diol), boronic acids (e.g., 4-vinylboronic acid), sulfonic acids (e.g., 4-vinylsulfonic acid), metal chelators (e.g., styrene iminodiacetic acid), acrylamides and derivatives (e.g., N-methyl acrylamide), methacrylamides and derivatives (e.g., N,N-dimethyl acrylamide, N-(3-aminopropyl)methacrylamide), alkenes (e.g., 4-pentenoic acid, 3-chloro-1-phenyl-1-propene) (meth)acrylic acid anhydride and derivates (e.g., methacrylic anhydride), silicon-containing monomers (e.g., (3-methacryloxypropyl)trimethoxy silane, tetramethyldisiloxane), polyenes (e.g., isoprene, 3-hydroxy-3,7,11-trimethyl-1,6,10-dodecatriene), azides (e.g., 4-azido-2,3,5,6-tetrafluorobenzoic acid), thiols (e.g., allyl mercaptan).

4. A SERRS active bead according to claim 1 wherein said polymer shell(s) is/are formed from acrylate terminated or otherwise unsaturated urethanes, carbonates and epoxies or silicon-based monomers.

5. A SERRS active bead according to claim 1 wherein said polymer shell(s) comprises crosslinking agents that lend rigidity to the subject polymeric compounds such as di-, tri-, tetra- and penta-functional acrylates, methacrylates, acrylamides, vinyls, allyls, and styrenes.

6. A SERRS active bead according to claim 1 wherein the aggregated metal colloid comprises silver, gold, copper or metal alloy colloid surfaces.

7. A SERRS active bead according to claim 1 wherein the aggregated metal colloid comprises a naked metal or a metal oxide layer on a metal surface.

8. A SERRS active bead according to claim 1 wherein the aggregated metal colloid comprises an organic coating of citrate or of a polylysine or polyphenol polymer, to increase its sorptive capacity.

9. A SERRS active bead according to claim 1 wherein the aggregated metal colloid is in the form of aggregated metal colloid particle or particles.

10. A SERRS active bead according to claim 9 wherein the aggregated metal colloid particles have a cross section of about 4-50 nm, or about 25-36 nm.

11. A SERRS active bead according to claim 1 wherein the SERRS active bead has a cross-section of less than 1 μm.

12. A SERRS active bead according to claim 1 wherein the SERRS active bead comprises a plurality of SERRS active nanoparticles, said SERRS active nanoparticles comprising aggregated metal colloid and a SERRS active dye or dyes absorbed thereto, wherein each of said nanoparticles generate a substantially identical SERRS signal, or are provided with differing dyes or dye combinations so as to generate distinguishable SERRS signals.

13. A SERRS active bead according to claim 1 wherein the outermost polymer shell further comprises functional groups selected from any of the following: carboxylic acids, active esters of carboxylic acids, alcohols, amines, epoxides, chloromethyl styryl groups, vinly groups, thiol groups, maleimide and succinimide, or is further derivitised by a linking group or groups, such that target molecules can react and become bounded to the surface of the bead(s), or such that a moiety may react and become bounded to the surface of the bead(s) which moiety is capable of binding said target molecule.

14. SERRS active bead according to claim 1 wherein the beads are used as a labelling technology for protein and antibody detection.

15. A SERRS active bead according to claim 1 wherein molecular imprinting methods are applied to impart specific molecular recognition properties to the polymer shell via templating approaches.

16. A SERRS active bead according to claim 1 further comprising a magnetic particle or particles encapsulated within the polymer bead, for rendering the SERRS active bead magnetic.

17. A library of SERRS active beads according to claim 1 wherein each bead comprises a discernable SERRS signal, the discernable SERRS signal being obtained by varying the SERRS active dyes and/or relative levels of the dyes within each bead.

18. A method of preparing SERRS active beads comprising the steps of:
1) forming an aggregated metal colloid comprising a SERRS active dye absorbed thereto;
2) mixing the aggregated metal colloid with a monomer in a suitable solvent; and
3) effecting polymerisation of the monomer so as to form polymer beads encapsulating the aggregated metal colloid.

19. A method of preparing SERRS active beads comprising the steps of:
a) providing a monomer in a suitable solvent;
b) effecting polymerisation of the monomer so as to form polymer beads;
c) adding aggregated metal colloid to a solution comprising the polymer beads; and
d) heating the solution so as to cause the aggregated metal colloid to be taken up and become encapsulated by the polymer beads.

20. A method according to claims 18 or 19 wherein the polymer beads are further reacted so as to add functional or linking groups to an outer surface of the bead thereby allowing a molecule to be attached to the bead.

21. A method of detecting a target molecule in a sample, the method comprising the steps of:
a) providing a SERRS active bead(s) according to claim 1 wherein the bead has been generated so as to have a specifically identifiable SERRS signal and the surface of which has been modified so as to have attached thereto a moiety capable of binding said target molecule;
b) contacting said bead(s) with the sample so as to allow any of said target molecule in the sample to bind to said bead(s); and
c) detecting said target molecule by way of detecting the specifically identifiable SERRS signal obtained from a bead or beads being bound to said target molecule.

22. The method according to claim 21 wherein the target molecule is an antibody, antigen, receptor, ligand or nucleic acid molecule, such as a DNA molecule.

23. The method according to claim 22 wherein the moiety capable of binding said target molecule is an antigen, antibody, ligand, receptor or complementary nucleic acid respectively.

24. The method according to claim 21 wherein the bead(s) are magnetic such that the bead(s) may easily be separated and/or concentrated after step b).

25. A method of detecting a specific nucleic acid in a sample, comprising the steps of:
a) providing a SERRS active bead(s) according to claim 1 wherein the bead has been generated so as to have a specifically identifiable SERRS signal and the surface of which has been modified so as to have attached thereto a moiety capable of binding said target molecule wherein the moiety is an oligonucleotide capable of specifically hybridising with a target nucleic acid;
b) contacting said bead(s) with the sample so as to allow any of said target nucleic acid in the sample to bind to said bead(s);
c) contacting said target bound beads with a further labelled nucleic acid and said further labelled nucleic acid to specifically hybridise thereto;
d) optionally carrying out an amplification reaction and further optionally detecting if said amplification reaction has been successful;
e) contacting said labelled hybridised nucleic acid bond beads with a surface capable of binding said labelled moiety; and
f) detecting said target molecule by way of detecting the specifically identifiable SERRS signal obtained from said beads which have been effectively bound to said substrate.

26. The method according to claim 25 further comprising one or more washing steps to remove unbound reagents from step(s) b, c and/or e to be removed.
